(19) United States
(12) Patent Application Publication
(10) Pub. No.: US 2021/0228232 A1
(43) Pub. Date: Jul. 29, 2021

(54) Cannula, in Particular for Lumbar Puncture and Lumbar Injection, as Well as Method of Production of Same

(71) Applicant: Pajunk GmbH Medizintechnologie, Geisingen (DE)

(72) Inventors: Simone Pajunk-Schelling, Geisingen (DE); Martin Hauger, Donaueschingen (DE)

(73) Assignee: Pajunk GmbH Medizintechnologie, Geisingen (DE)

(21) Appl. No.: 17/160,114

(22) Filed: Jan. 27, 2021

(30) Foreign Application Priority Data
Jan. 29, 2020 (DE) 10 2020 102 119.4

(51) Int. Cl. A61B 17/34

(52) U.S. Cl. CPC A61B 17/3421; A61B 2017/00526

(57) ABSTRACT

A cannula, in particular for lumbar puncture and lumbar injection, with a proximal end and a distal end, can include a cannula tube with an inner side, and outer side, a cannula canal having a first end region and a second end region. The cannula tube can include at the second end region a tip which forms the distal end of the cannula, and the cannula tube can include an opening of the cannula canal, and a coating is provided about the opening of the cannula canal on the inner side.

### CROSS-REFENCE TO RELATED APPLICATIONS

This application claims priority to German Patent Application No. 10 2020 102 119.4, filed Jan. 29, 2020, which is incorporated by reference in its entirety.

### BACKGROUND

The application relates to an improved cannula, in particular for lumbar puncture and lumbar injection, as well as to a production method of an improved cannula.

### SUMMARY

Prior art has disclosed cannulas in various implementations which typically comprise a proximal end and a distal end and a cannula tube, wherein at the distal end an opening is provided.

In spinal anesthesia, anesthetics are injected by means of cannulas of the generic kind into the spinal canal of the vertebral column for the purpose of blocking nerves. Using lumbar puncture, cerebrospinal fluid can be withdrawn for the diagnosis of certain diseases or radiopaque substances or medicinal drugs can be injected into the spinal canal.

Early preventative examinations allow Alzheimer's disease and also dementia to be detected and promising results can be obtained using appropriate drugs following early diagnosis. However, a precondition requires completing regular early examinations in which cerebrospinal fluid is withdrawn from the spinal canal for diagnostic purposes.

In the past, cannulas have been found to be useful for these tests, which comprise a closed and conical tip whose opening for the removal or injection is located laterally behind the tip. These cannulas are also known in prior art as Sprotte® cannulas as disclosed in DE 3 020 926 A1 and are distinguished thereby that in a puncture or injection into the tough membrane, or dura mater, of the spinal canal an atraumatic penetration is ideally accomplished and thus a minimal puncture hole is formed whereby undesirable leaking of cerebrospinal fluid can be minimized. A loss of cerebrospinal fluid can lead to pressure decrease in the entire cerebrospinal fluid space and thereby to diffuse side effects such as, for example, headaches. A significant pressure decrease can even lead to temporary cranial nerve paralysis or to tears in the blood vessels of the dura mater.

Alternatively, cannulas can be utilized whose tips as the distal ends are developed through oblique beveling of the cannula tube.

A disadvantage of the prior known cannulas has been found to be that during the production of the prior known cannulas for spinal puncture extremely extensive cleaning is required in order to ensure that subsequent to the production process no contaminant residues remain on the cannula tube and especially also not within the cannula tube.

The present application proposes remedies to counteract the above described disadvantage.

The present application therefore addresses the problem of providing an improved cannula for lumbar puncture and lumbar injection which advantageously improves the prior known cannulas and can be provided employing a production method which, at identical or improved functionality of the cannulas, combines economic advantages. The improved cannula is to increase, in particular, the safety of the patient.

These problems are resolved through a cannula with the characteristics disclosed herein and a method for the production of a cannula for lumbar puncture and lumbar injection with the characteristics disclosed herein.

The cannula, in particular for lumbar puncture and lumbar injection with the characteristics has a proximal end and a distal end and comprises furthermore a cannula tube with an inner side, an outer side, a first end region and a second end region, wherein the cannula tube forms a cannula canal. In addition, the cannula tube comprises at the second end region a tip forming the distal end of the cannula, wherein in the cannula tube an opening of the cannula canal is provided and wherein in the cannula canal on the inner side a coating is provided at least about the opening.

The application is based on the concept that, after working in the opening, which can be produced for example using a machining process, an erosion process or by laser cutting, a coating is applied at least about the opening on the inner side of the cannula tube. The coating can ensure that contaminants or residues remaining even after technical cleaning of the inner side of the cannula tube, are imbedded within it, whereby the cannula is especially clean for the examinations to be carried out and the safety of the patient is assured.

The coating prevents furthermore that material, for example in the form of metal ions, can become detached from the cannula tube and are entrained in the fluid flowing through the cannula tube. The coating can, in addition, accomplish optimization and/or equalization of the surface.

According to a further embodiment, the coating covers an internal edge of the opening. The opening is typically worked into the cannula tube by the manufacturing process of grinding and it has been found that the coating of the internal edge can imbed contaminations and lead as well also to the reduction of roughnesses due to fabrication and of sharp-edged regions.

It has moreover been found to be advantageous for the coating on the outer side to be disposed at least about the opening. It is in particular preferred for the coating on the inner side of the cannula tube, at least about the opening and the coating on the outer side of the cannula tube at least about the opening, are connected with one another through the opening across the internal edge, whereby a unitary coherent coating can be provided which can imbed all contaminants in the region of the opening.

A further advantageous embodiment provides that the inner side and/or the outer side of the cannula tube is or are completely covered by the coating. The layer thickness of the coating can vary over the discrete regions of the cannula tube, wherein the layer thickness on the inner side as well as also on the outer side of the cannula tube should be kept as thin as possible in order not to enlarge unnecessarily the diameter of the cannula tube on the outer side and not to decrease said diameter on the inner side. The cross section of the cannula tube through which flow can pass, furthermore, should be dimensioned so as to be adequate for a mandrin to be inserted into the cannula tube to prevent the penetration of tissue into the cannula tube during the introduction of the cannula. According to a preferred further development, the cannula comprises a mandrin which also, at least in sections, preferably completely, is provided with a coating.

The tip is preferably closed and approximately conically developed and the opening is disposed adjacently to the tip of the cannula tube. An atraumatic cannula can be provided through such an implementation with the use of which it can be avoided that during penetration tissue penetrates into the opening.

According to a further advantageous implementation of the present cannula, the opening can be elongated in a longitudinal direction of the cannula tube. The longitudinal direction of the cannula tube is determined by the direction of the longest extent of the cannula tube, or stated differently, through the vector between the distal end and the proximal end of the cannula tube. By elongated opening of the cannula tube is here and subsequently to be understood an opening which is longer in the longitudinal direction than transversely to the longitudinal direction, preferably at least at a ratio of 1.5:1.

An alternative preferred further development provides for the cannula tube comprising at the second end region an oblique beveling through which the tip is formed and the opening is disposed at the front end in the second end region of the cannula tube.

A further advantageous embodiment provides for a (necked) extension to be provided on the proximal end of the cannula. The extension can comprise a connection by which the extension can be connected with a tube or a syringe. The connection can preferably be developed, for example, as a connector, in particular as a Luer lock connector or as a NRFIT® connector.

It has furthermore been found to be advantageous for the coating to sheath the extension also. In a preferred embodiment, the coating can encompass the cannula completely such that the inner side as well as also all outer surfaces of the cannula are provided with a coating whereby all contaminants are imbedded.

The cannula tube preferably has an outer diameter in the range of 0.2 to 1.5 mm, preferably in the range of 0.4 to 1.2 mm, an inner diameter in the range of 0.15 to 1.4 mm, and/or a length in the range of 90 to 500 mm, preferably of 90 to 300 mm, especially preferred of 90 to 120 mm.

The coating has preferably a thickness in the range of 0.1 to 50 μm, preferably in the range of 0.1 to 30 μm, especially preferred in the range of 0.1 to 15 μm.

A further aspect of the relates to a production method of a cannula, in particular of a cannula for lumbar puncture and lumbar injection, wherein the production method comprises the following method steps:
providing the cannula tube with an inner side, an outer side, an open first end region and a second end region,
working in an opening, and
coating the inner side of the cannula tube at least about the opening.

It is preferably provided that either the second end region comprises a tip which is closed and developed approximately conically, and that the opening is worked in adjacently to the tip, or that the cannula tube is worked in at the second end region with an oblique beveling through which a tip is formed, and that the opening is disposed on the front end on the cannula tube in the second end region.

A further development of the method provides that the entire inner side of the cannula tube or the entire inner side and the entire (exposed) outer side of the cannula tube is or are coated. By exposed outer side of the cannula tube is here understood the region of the cannula tube which is, for example, not obscured by the extension.

An advantageous further development of the present method provides for the opening into the cannula tube to be worked in through a machining production method, in particular through grinding, alternatively through erosion or laser cutting. Using these production methods, the opening can be worked into the cannula tube without incurring the risk of generating undesirable deformations on the cannula tube.

It has, moreover, been found to be advantageous if an extension is formed onto the cannula tube on the first end region before the coating, wherein preferably the extension is formed onto the cannula tube by injection molding.

According to a further development, the inner side of the cannula at least in regions can be coated at least around the opening. In addition, further preferred is that the entire inner side of the cannula tube can be coated. It can in particular be advantageous if a distal and closed end of the cannula canal or the “dead” end in the cannula canal is closed in the second end region of the cannula tube between the opening and the closed end region.

It has also been furthermore found to be advantageous for the internal edge of the opening to be coated and furthermore at least the outer side of the cannula tube about the opening. In an especially preferred embodiment, all surfaces of the cannula, after the opening has been worked in, are provided with the coating such that all possible contaminants on the cannula are imbedded and an especially clean cannula can be provided.

A further aspect relates to the use of a cannula for lumbar puncture and lumbar injection.

### BRIEF DESCRIPTION OF THE DRAWINGS

With reference to the accompanying drawing, an embodiment example of a cannula, in particular for lumbar puncture and lumbar injection, will be described in detail. Therein depict:

FIG. 1a a schematic, partially sectioned and highly simplified representation of a first embodiment example of a cannula for lumbar puncture and lumbar injection,

FIG. 1b an enlarged representation of a distal end of the cannula with a conical and closed tip,

FIG. 2 a schematic, partially sectioned and highly simplified representation of a mandrin of a cannula for lumbar puncture and lumbar injection according to FIG. 1,

FIG. 3a a schematic, partially sectioned and highly simplified representation of a second embodiment example of a cannula for lumbar puncture and lumbar injection,

FIG. 3b an enlarged representation of a distal end of the cannular with a tip formed by an oblique beveling,

FIG. 4 a schematic, partially sectioned and highly simplified representation of a mandrin of a cannula for lumbar puncture and lumbar injection according to FIG. 3.

### DETAILED DESCRIPTION

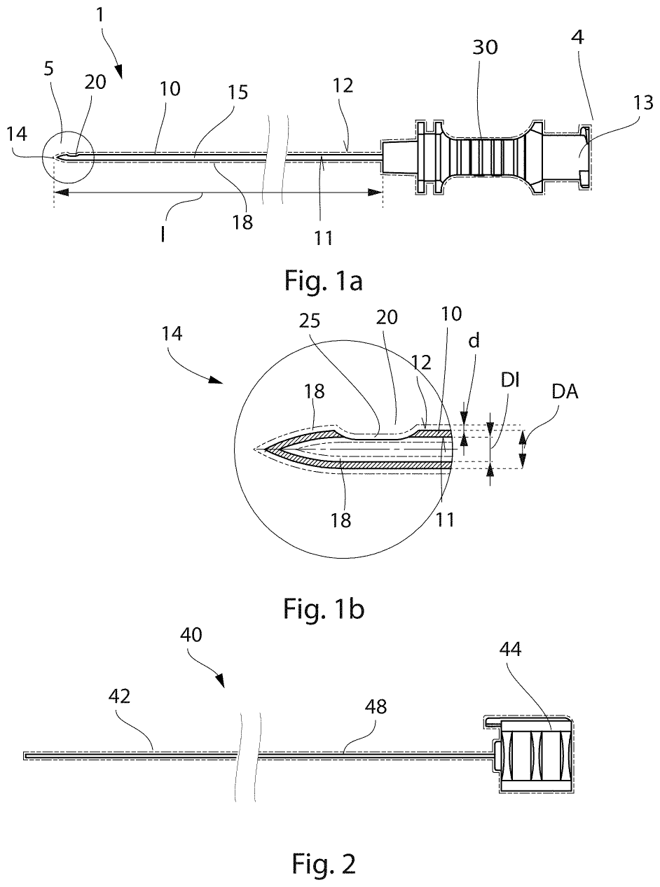

FIG. 1a shows a cannula 1 for lumbar puncture and lumbar injection which can be utilized to inject an anesthetic substance into the spinal canal of the vertebral column of a patient. The cannula 1 depicted in FIG. 1a can furthermore be utilized for diagnostic purposes or for early detection examinations to withdraw cerebrospinal fluid from the spinal canal of a patient or to inject a radiopaque substance into the spinal canal.

The cannula 1 has a proximal end 4 and a distal end 5 and comprises a cannula tube 10 and a (necked) extension 30.

The cannula tube 10 can be produced of a metal material or a synthetic material, for example of a carbon fiber-reinforced synthetic material. The cannula tube 10 can have an outer diameter DA in the range of 0.2 to 1.5 mm, preferably in the range of 0.4 to 1.2 mm. The cannula tube 10 can furthermore have an inner diameter DI in the range of 0.15 to 1.4 mm. Lastly, the cannula tube 10 can have a length l in the range of 90 to 500 mm, preferably of 90 to 300 mm, especially preferred of 90 to 120 mm.

The cannula tube 10 comprises an open first end region and, according to the first embodiment example depicted in FIGS. 1a and 1b, a closed second end region. The cannula tube 10 comprises an inner side 11 and an outer side 12, wherein the inner side 11 forms the wall of a lumen or of the cannula canal 15. The second end region of the cannula tube 10 comprises a conical or cone-shaped tip 14 that forms the distal end 5 of the cannula 1.

Adjacent to the tip 14 of the cannula tube 10, which is depicted in detail in FIG. 1b, the opening 20 is disposed with an internal edge 25 surrounding the opening 20, through which the cannula canal 15 is accessible from a radial direction.

The spacing between the opening 20 and the distal end 4 or the tip 14 is selected such that the opening 20 is developed completely on a lateral surface of the cannula tube 10.

Onto the proximal end 4 the extension 30 is formed onto the cannula tube 10, wherein, preferably the extension 30 forms a connection on which a tube or a syringe can be secured. The connection can be developed as a Luer lock connector or as an NRFit® connector.

As is evident in FIGS. 1a and 1b, the cannula 1 comprises a coating 18 depicted in dashed lines. The coating 18 is applied onto the inner side 11 and/or the outer side 12 of the cannula tube 10 around the opening 20 and closes the cannula canal 15 between the opening 20 and the distal end 5. The coating 10—as depicted—can furthermore extend completely over the internal edge 25 of the opening 20 whereby a unitary or monolithic coating 18 can be formed. The coating [18] preferably covers completely and non-intermittently all surface areas of the cannula tube 10 or of the cannula 1 and preferably imbeds possible contaminants on the surfaces, whereby these are rendered harmless. The coating 18 is preferably diffusion-tight and/or abrasion resistant.

For the production of the cannula according to FIG. 1, first, the cannula tube 10 is preferably provided as a discrete part and subsequently the extension 30 can be formed onto the open first end region of the cannula tube 10, using, for example injection molding. In a subsequent step, the opening 20 can be worked into the cannula tube 10 in the second end region adjacent to the tip 14, wherein the opening 20 preferably can be worked into the cannula tube 10 as an elongated opening by a machining fabrication method, in particular by grinding.

After the opening [20] has been worked into the cannula tube 10, the already functionable cannula 1 can be cleaned by a cleaning method in order to remove contaminants due to the working-in of the opening 20 and the other production steps. To ensure that even after the cleaning, in particular at sites difficult of access, such as, for example, within the closed and conical tip 14 at the distal end 5 of the cannula 1 or in a “dead” end between the opening 20 and the closed second end region of the cannula tube 10, no contaminants remain, a coating 18, which is depicted in the accompanying Figures in dashed lines, can be applied over the entire surface on all inner surfaces as well as outer surfaces.

The coating 18 can embed or agglutinate all contaminants which may be located in the cannula tube 10 and on the extension [30].

The coating 18 can preferably be applied using a thin-layer method and can be formed of a synthetic material as well as also of a metal material. A thickness d of the coating can be in the range of 0.1 to 50 μm, preferably in the range of 0.1 to 30 μm especially preferred in the range of 0.1 to 15 μm.

FIG. 2 shows a mandrin 40, comprising a rod section 42 and an extension section 44, wherein the rod section 32 [sic: 42] is introducible into the lumen or the cannula canal 15 and, in the inserted state, stabilizes the cannula tube 10. The mandrin 40 can close the opening during the penetration and prevent tissue from entering which could plug up the cannula tube 15 [sic: 10]. The mandrin 40, in particular the rod section 42, can comprise a metal core or a core of a synthetic material, for example a carbon fiber-reinforced synthetic material, while the extension* section 44, analogous to the (necked) extension 30 of the cannula 1, can be formed onto the core. The mandrin 40 can furthermore comprise a coating 48 which fully covers the mandrin 40. The material and the coating method of the coatings 18, 48 of cannula 1 and of mandrin 40 can be identical.

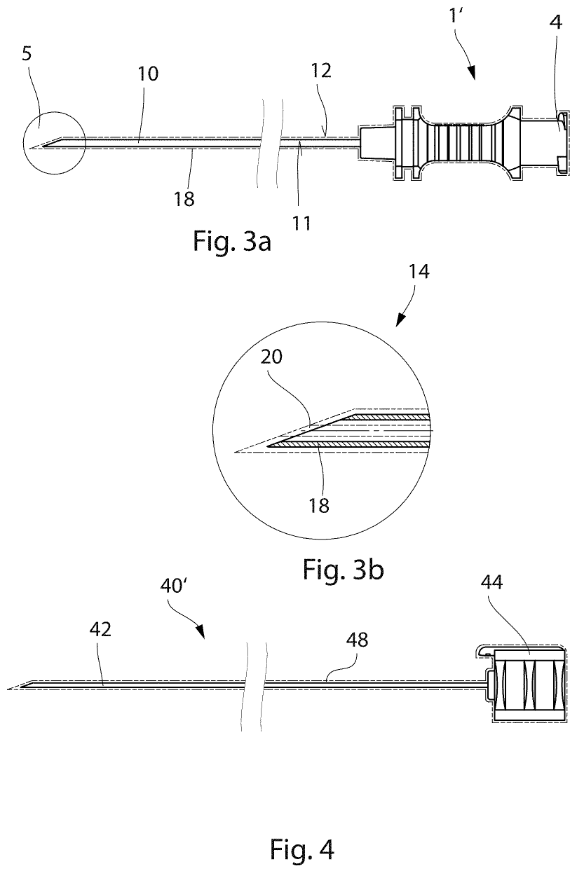

The second embodiment example of a cannula 1′, depicted in FIGS. 3a and 3b, as well as the second embodiment example of a mandrin 40′, depicted in FIGS. 4, differ from the cannula 1, depicted in FIGS. 1a and 1b, or the mandrin 40, depicted in FIG. 2, only in the embodiment of the distal end. The additional explanations regarding cannula 1 and mandrin 40 are transferable to cannula 1′ and to mandrin 40′.

The cannula tube 10 comprises an open first end region and an open second end region. The second end region of the cannula tube 10 comprises herein an oblique beveling through which the tip 14 is formed which forms the distal end 5 of the cannula 1. The opening 20 in this embodiment example is not developed on the lateral surface of the cannula tube 10 but rather at the front end in the second end region of the cannula tube 10.

Cannula 1′ also comprises a coating 18 shown in dashed lines. The coating 18 is applied on the inner side 11 and/or on the outer side 12 of cannula tube 10 about the opening 20. The coating 18—as depicted—can moreover extend completely over the internal edge 25 of opening 20, whereby a unitary or monolithic coating 18 can be formed. The coating [18] preferably covers completely and non-intermittently all surface areas of the cannula tube 10 or of cannula 1.

Cannula 1′ can also comprise a mandrin 40′ which is depicted in FIG. 4 and differs from the mandrin 40 depicted in FIG. 2 only therein that a distal end of the mandrin 40′ is developed to be slanted.

### LIST OF REFERENCE SYMBOLS

1, 1′ Cannula

4 Proximal end

5 Distal end

10 Cannula tube

11 Inner side

12 Outer side

[13 on FIG. 1a, unused]

14 Tip

15 Cannula canal

[18 Coating]

20 Opening

25 Internal edge

30 Extension

40, 40′ Mandrin

42 Rod section

44 Extension section

48 Coating

d Thickness

DA Outer diameter

DI Inner diameter

l Length

1. A cannula comprising:
a cannula tube with an inner side an outer side, and a cannula canal formed of a first end region and a second end region,
a tip of the cannula tube located at the second end region and which forms a distal end of the cannula,
an opening of the cannula canal of the cannula tube, and
wherein a coating is provided in the cannula canal on the inner side about the opening.

2. The cannula as in claim 1, wherein the coating covers an internal edge of the opening.

3. The cannula as in claim 1, wherein the coating is disposed on the outer side about the opening.

4. The cannula as in claim 3, wherein the coating on the inner side of the cannula tube and on the outer side of the cannula tube is connected through the opening.

5. The cannula and in claim 1, wherein the coating completely covers the inner side of the cannula tube, the outer side of the cannula tube, or both the inner side of the cannula tube and the outer side of the cannula tube.

6. The cannula as in claim 1, wherein the tip is closed and approximately conically and the opening is disposed adjacently to the tip of the cannula tube.

7. The cannula as in claim 6, wherein the opening is elongated in a longitudinal direction of the cannula tube.

8. The cannula as in claim 1, wherein the cannula tube comprises an oblique beveling by which the tip is formed and the opening is disposed at a front end in the second end region of the cannula tube.

9. The cannula as in claim 1, wherein an extension is provided at the proximal end.

10. The cannula as in claim 9, wherein the coating sheathes the extension.

11. The cannula as in claim 1, wherein the coating is unitary or monolithic.

12. The cannula as in claim 1, further comprising a mandrin which comprises a second coating.

13. The cannula as in claim 1, wherein the cannula tube has an outer diameter (DA) in the range of 0.2 to 1.5 mm, an inner diameter (DI) in the range of 0.15 to 1.4 mm and a length (l) in the range of 90 to 500 mm, or a combination thereof.

14. The cannula as in claim 1, wherein the coating has a thickness (d) in the range of 0.1 to 50 μm.

15. A method of producing a cannula, comprising:
providing a cannula tube with an inner side, an outer side, and a cannula canal formed of a open first end region and a second end region,
machining an opening of the cannula canal of the cannula tube, and
coating an inner side of the cannula tube at least about the opening.

16. The method as in claim 15, wherein the second end region comprises a tip which is closed and developed approximately conically and the opening is worked in adjacently to the tip.

17. The method as in claim 15, wherein the cannula tube is worked at the second end region with an oblique beveling through which a tip is formed and the opening is disposed at the front end in the second end region of the cannula tube.

18. The method as in claim 15, further comprising coating the entire inner side or the entire inner side and outer side of the cannula tube.

19. The method as in claim 15, wherein before the coating an extension is formed onto the cannula tube at the first end region by an injection molding process.
